(19) United States
(12) Patent Application Publication
(10) Pub. No.: US 2021/0299629 A1
(43) Pub. Date: Sep. 30, 2021

(54) ARRAY OF POLYMERIC HYDROGEL NANOSTRUCTURES AND THEIR USES

(71) Applicant: CORNING INCORPORATED, CORNING, NY (US)

(72) Inventor: Ye Fang, Painted Post, NY (US)

(21) Appl. No.: 17/265,307

(22) PCT Filed: Aug. 1, 2019
(86) PCT No.: PCT/US2019/044629

§ 371 Date: Feb. 2, 2021

Related U.S. Application Data
(60) Provisional application No. 62/715,075, filed on Aug. 6, 2018.

(51) Int. Cl. B01J 19/00; B01L 3/00; C12Q 1/6874

(52) U.S. Cl. CPC B01J 19/0046; B01L 3/502707; C12Q 1/6874; B01J 2219/00317; B01L 2300/12; B01J 2219/00644; B01L 2300/0893; B01L 2300/0896; B01L 2200/12; B01J 2219/00527

(57) ABSTRACT

A method for making a microfluidic device having one or more different patterned polymeric hydrogel nanostructure is provided. The method includes: providing a first substrate having a first patterned array of polymeric hydrogel nanostructures on a first interior surface and a peripheral surface portion; providing a second substrate having a second interior surface and a side wall with an end surface; and bonding the end surface of the second substrate to the peripheral surface portion of the first substrate such that the first and second interior surfaces define a hermetic cavity within the bonded first and second substrate. The microfluidic device can be designed to include a variety of different patterned array of polymeric hydrogel nanostructures depending on the desired application and properties for the device.

### CROSS-REFERENCE TO RELATED APPLICATIONS

This application claims the benefit of priority under 35 U.S.C. § 119 of U.S. Provisional Application No. 62/715,075, filed Aug. 6, 2018, the content of which is incorporated herein by reference in its entirety.

### FIELD

This disclosure is related to patterned surfaces and microfluidics devices, methods of manufacturing patterned microfluidic devices, and methods of using such devices for biomolecular analysis, in particular gene sequencing.

### BACKGROUND

Biological samples can often be complicated in composition and amount. Analysis of biomolecules in a biological sample often involves partitioning a sample into thousands or even millions of samples for quantitative determination. Many different partitioning methods have been developed, including surface patterning (including surface chemistry and structure patterning), microdroplets, continuous or discontinuous flow, and separation under physical force (e.g., electrophoresis). Among them, surface patterning is one of the most common and effective means to selectively capture and partition biomolecules in a biological sample for bioanalysis. Furthermore, owing to its ability to spatially and/or temporally control bioreactions, microfluidics has been combined with surface patterning to achieve high sensitivity and specificity for biomolecular analysis. For instance, for optical detection based massively parallel gene sequencing applications, millions of short DNA fragments generated from a genomic DNA sample can be captured and partitioned onto a patterned surface of a microfluidic device such that these corresponding DNA fragments are spatially separated from each other to facilitate sequencing by, for example, synthesis, ligation, or single-molecule real-time imaging. These gene sequencing techniques can be used to sequence entire genome, or small portions of the genome such as the exome or a preselected subset of genes.

Several approaches have been used to fabricate nanopatterned substrates for biomolecular analysis, in particular for massively parallel gene sequencing applications. Conventional methods make patterned substrates using photolithography and nanoimprinting since these techniques have high throughput and high fidelity in making patterns including nanofeatures on a solid surface. Photolithography is generally useful for patterning flat wafer substrates (e.g., glass, and silicon), while nanoimprinting can be applied in patterning flat or curved wafer substrates.

A variety of massively parallel gene sequencing techniques that are divergent in DNA immobilization chemistry, clustering, and DNA sequencing principles have been developed in the past decades. For instance, for sequencing using bridge amplification or template walking, DNA molecules are covalently captured and partitioned onto a flat substrate having a polymeric hydrogel coating or a short linker molecule, respectively. For sequencing using exclusion amplification, DNA molecules are selectively captured and partitioned on a patterned nanowell substrate having a polymeric hydrogel coating. For sequencing by ligation, DNA nanoballs generated using a rolling circle replication amplification are electrostatically captured onto a patterned positively charged surface (e.g., amine silane coated surface). For sequencing using single molecule detection, DNA molecules are covalently attached to a substrate surface. Unfortunately, each of these techniques can be more time consuming, supply demanding, and/or complicated to perform the analysis and interpret results.

Accordingly, there is a need for improved techniques and corresponding surface chemistries to improve patterned surfaces in microfluidic devices for more precise and cost effective gene sequencing applications.

### SUMMARY OF THE DISCLOSURE

According some aspects of the present disclosure, a method for making a periodic array of polymeric nanodimples is provided. The method includes: providing a substrate; priming the substrate with a priming molecule to form a primed substrate; spin coating the primed substrate with a mixture of hydrogel monomers and silica nanoparticles to form a coated substrate; exposing the coated substrate with UV irradiation to form a polymeric hydrogel; removing a portion of the polymeric hydrogel to partially expose the silica nanoparticles; depositing a metal layer, a metal oxide layer, or a combination of both to a top surface of the polymeric hydrogel; and etching away the silica nanoparticles to form a periodic array of polymeric hydrogel nanodimples having metal or metal oxide regions on the top surface of the polymeric hydrogel.

According to other aspects of the present disclosure, a method for making a periodic array of polymeric nanoposts is provided. The method includes: providing a substrate; priming the substrate with a priming molecule to form a primed substrate; spin coating the primed substrate with a mixture of hydrogel monomers and silica nanoparticles to form a coated substrate; exposing the coated substrate with UV irradiation to form a polymeric hydrogel; removing portions of the polymeric hydrogel to partially expose the silica nanoparticles and substrate; and etching away the silica nanoparticles to form a periodic array of polymeric hydrogel nanoposts.

According to yet other aspects of the present disclosure, a method for making a periodic array of polymeric nanoposts inside nanowells is provided. The method includes: providing a substrate; priming the substrate with a priming molecule to forma primed substrate; spin coating the primed substrate with a mixture of hydrogel monomers and silica nanoparticles to form a coated substrate; exposing the coated substrate with UV irradiation to form a polymeric hydrogel; removing portions of the polymeric hydrogel to partially expose the silica nanoparticles and substrate; depositing a metal layer, a metal oxide layer, or a combination thereof; and etching away the silica nanoparticles to form a periodic array of polymeric hydrogel nanoposts enclosed inside metal or metal oxide nanowells.

According to still other aspects of the present disclosure, a method for making a periodic array of polymeric nanoposts surrounded by a metal or oxide ring is provided. The method includes: providing a substrate; priming the substrate with a priming molecule to form a primed substrate; spin coating the primed substrate with a mixture of hydrogel monomers and silica nanoparticles to form a coated substrate having a monolayer of non-close-packed colloidal crystals; exposing the coated substrate with UV irradiation to form a polymeric hydrogel; removing portions of the polymeric hydrogel to partially expose the silica nanoparticles and substrate; depositing a metal layer, a metal oxide layer, or a combination thereof; ion beam etching away a portion of the metal or metal oxide layer to form a metal or metal oxide ring; and etching away the silica nanoparticles to form a periodic array of polymeric hydrogel nanoposts enclosed by the metal or metal oxide ring.

According to other aspects of the present disclosure, a method for making a microfluidic device having a patterned polymeric hydrogel nanostructure is provided. The method includes: providing a first substrate having a first patterned array of polymeric hydrogel nanostructures on a first interior surface and a peripheral surface portion; providing a second substrate having a second interior surface and a side wall with an end surface; and bonding the end surface of the second substrate to the peripheral surface portion of the first substrate such that the first and second interior surfaces define a hermetic cavity within the bonded first and second substrate.

According to other aspects of the present disclosure, a method for functionalizing a microfluidic device having a patterned polymeric hydrogel nanostructure using a primer DNA is provided. The method includes: providing a microfluidic device having at least one channel floor surface having a patterned polymeric hydrogel nanostructure; and incubating the microfluidic device with a primer DNA to covalently attach the primer DNA to the channel floor surface.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments.

### BRIEF DESCRIPTION OF THE DRAWINGS

The following is a description of the figures in the accompanying drawings. The figures are not necessarily to scale, and certain features and certain views of the figures may be shown exaggerated in scale or in schematic in the interest of clarity and conciseness.

FIG. 1 is a schematic view of a patterned substrate according to some aspects of the present disclosure;

FIG. 2 is a flow chart demonstrating a method for making a periodic array of polymeric nanodimples according to some aspects of the present disclosure;

FIG. 3 is a flow chart demonstrating a method for making a periodic array of polymeric nanoposts according to some aspects of the present disclosure;

FIG. 4 is a flow chart demonstrating a method for a method for making a periodic array of polymeric nanoposts and a method for making a periodic array of polymeric nanoposts inside nanowells and a periodic array of polymer nanoposts surrounded by a ring of a metal material, according to some aspects of the present disclosure;

FIG. 5 is a flow chart demonstrating a method for making a microfluidic device having a patterned polymeric hydrogel nanostructure according to some aspects of the present disclosure;

FIG. 6 is a schematic top view of a microfluidic device according to some aspects of the present disclosure; and

FIG. 7 is a schematic cross-sectional view of the microfluidic device taken along line 7-7 of FIG. 6.

### DETAILED DESCRIPTION

Additional features and advantages will be set forth in the detailed description which follows and will be apparent to those skilled in the art from the description, or recognized by practicing the embodiments as described in the following description, together with the claims and appended drawings.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

In this document, relational terms, such as first and second, top and bottom, and the like, are used solely to distinguish one entity or action from another entity or action, without necessarily requiring or implying any actual such relationship or order between such entities or actions.

Modifications of the disclosure will occur to those skilled in the art and to those who make or use the disclosure. Therefore, it is understood that the embodiments shown in the drawings and described above are merely for illustrative purposes and not intended to limit the scope of the disclosure, which is defined by the following claims, as interpreted according to the principles of patent law, including the doctrine of equivalents.

For purposes of this disclosure, the term “coupled” (in all of its forms: couple, coupling, coupled, etc.) generally means the joining of two components directly or indirectly to one another. Such joining may be stationary in nature or movable in nature. Such joining may be achieved with the two components and any additional intermediate members being integrally formed as a single unitary body with one another or with the two components. Such joining may be permanent in nature, or may be removable or releasable in nature, unless otherwise stated.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

Directional terms as used herein—for example up, down, right, left, front, back, top, bottom—are made only with reference to the figures as drawn and are not intended to imply absolute orientation.

As used herein the terms “the,” “a,” or “an,” mean “at least one,” and should not be limited to “only one” unless explicitly indicated to the contrary. Thus, for example, reference to “a component” includes embodiments having two or more such components unless the context clearly indicates otherwise.

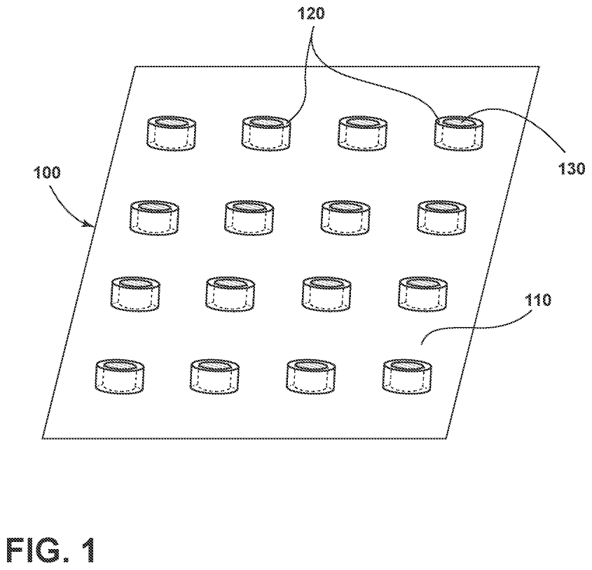

Referring to FIG. 1, a schematic view of a patterned substrate 100 is provided according to some aspects of the present disclosure. The microfluidic devices disclosed herein having at least one patterned substrate surface, wherein the pattern includes an array of polymeric hydrogel nanostructures made using templated silica nanosphere lithography. The microfluidic devices may contain at least one fluidic channel. In some aspects, the microfluidic device also contains at least one inlet port and one outlet port for each channel. The channel and inlet/outlet ports can be made on the pattered substrate 100 or another substrate. In some aspects, as illustrated in FIG. 1, the array of polymeric hydrogel nanostructures positioned on the patterned substrate 100 may include an array of polymeric hydrogel nanoposts 130 where each nanopost 130 can be surrounded by a metal or metal oxide ring 120. A surface 110 of the patterned substrate may be exposed at interstitial regions between nanoposts 130. Although nanoposts 130 are shown, the type of polymeric hydrogel nanostructures and their respective geometries, shapes, and dimensions can be varied depending on the given application or desired properties to be provided. For example, in some aspects, the polymeric hydrogel nanostructures may include, but are not limited to, nanoposts, nanodimples, nanocones, nanocubes, nanocylinders, nanododecahedrons, nanotorus, nanocuboids, nanospheres, nanotetrahedrons, nanoicosahedrons, nanoellipsoids, nanohexagonal pyramids, nanotriangular prisms, nanooctahedron, nanopentagonal prisms, nanohemispheres, nanohexagonal prisms, nanoparallelepiped, nanopentagonal prism, or any other 3-D shape or structure on a nanometer, micrometer, and/or millimeter scale know in the art. In addition, in some aspects, the list of polymeric hydrogel nanostructures provided herein may have a metal or metal oxide ring, metal or metal oxide coating, metal or metal oxide layer, and/or metal or metal oxide surface applied to the respective polymeric hydrogel nanostructures. As detailed herein, in some aspects, the array of polymeric hydrogel nanostructures may include nanodimples separated by intestinal, or interstitial, metal or metal oxide regions on the top of the polymeric hydrogel films, nanoposts separated by bare substrate surface, and/or nanoposts enclosed inside a metal or metal oxide nanowell.

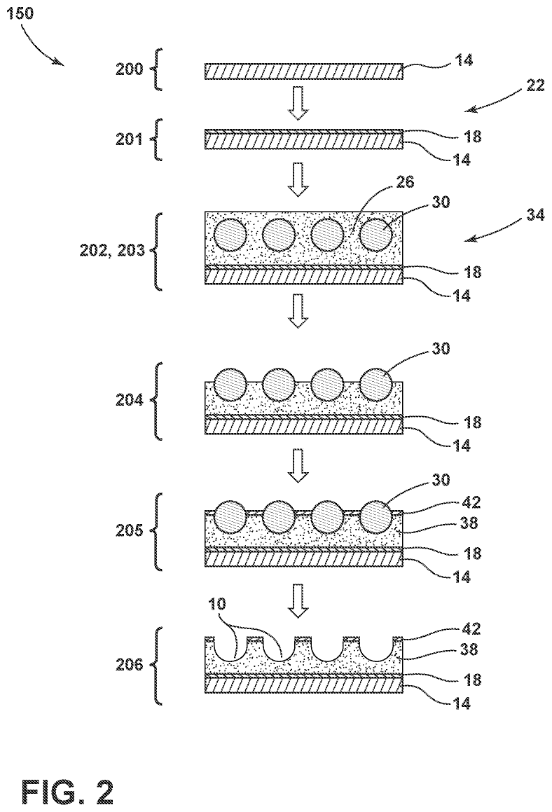

Referring now to FIG. 2, a method 150 for making a periodic array of polymeric hydrogel nanodimples 10 is provided according to some aspects of the present disclosure. The method 150 includes: providing a substrate 14 (step 200); priming the substrate 14 with a priming molecule 18 to form a primed substrate 22 (step 201); coating the primed substrate 22 with a mixture of hydrogel monomers 26 and nanoparticles 30 (e.g., depositing the mixture of hydrogel monomers and nanoparticles on the primed substrate by spin coating or another suitable deposition process) to form a coated substrate 34 (step 202); exposing the coated substrate 34 (e.g., irradiating the coated substrate with ultraviolet (UV) and/or another suitable wavelength irradiation) to form a polymeric hydrogel 38 (step 203); removing a portion of the polymeric hydrogel 38 to partially expose the nanoparticles 30 (step 204); depositing a metal layer 42, a metal oxide layer 42, or a combination thereof to a top surface of the polymeric hydrogel 38 (step 205); and etching away the nanoparticles 30 to form the periodic array of polymeric hydrogel nanodimples 10 having interstitial metal or metal oxide regions 42 on the top surface of the polymeric hydrogel 38 (step 206). The nanoparticles can comprise inorganic nanoparticles such as, for example, silica nanoparticles.

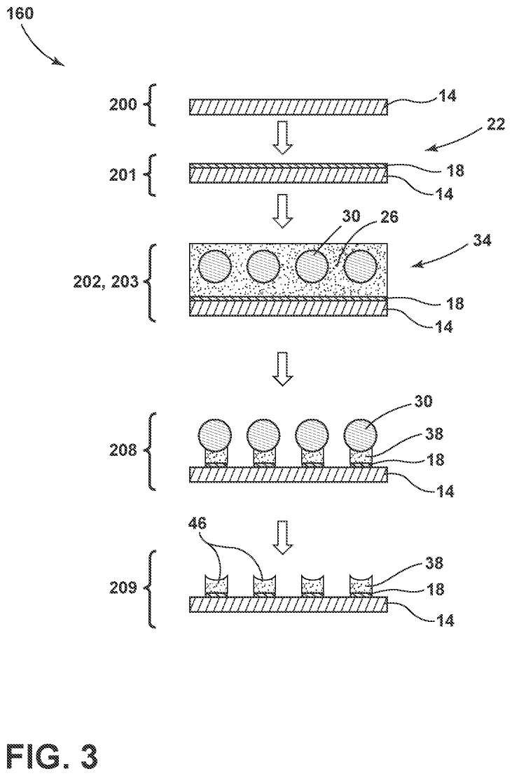

Referring to FIG. 3, a method 160 for making a periodic array of polymeric hydrogel nanoposts 46 is provided according to some aspects of the present disclosure. The method 160 includes: providing the substrate 14 (step 200); priming the substrate 14 with the priming molecule 18 to form the primed substrate 22 (step 201); coating the primed substrate 22 with a mixture of hydrogel monomers 26 and nanoparticles 30 (e.g., depositing the mixture of hydrogel monomers and nanoparticles on the primed substrate by spin coating or another suitable deposition process) to form the coated substrate 34 (step 202); exposing the coated substrate 34 (e.g., irradiating the coated substrate with UV and/or another suitable wavelength irradiation) to form the polymeric hydrogel 38 (step 203); removing portions of the polymeric hydrogel 38 to partially expose the nanoparticles 30 and substrate 14 (step 208); and etching away the nanoparticles 30 to form the periodic array of polymeric hydrogel nanoposts 46 (step 209). The nanoparticles can comprise inorganic nanoparticles such as, for example, silica nanoparticles. Each hydrogel nanopost 46 can comprise a polymeric hydrogel nanodimple as shown in FIG. 3. For example, the polymeric hydrogel nanodimple can be disposed at a top surface of the hydrogel nanopost 46. The surface of the substrate 14 (e.g., the substrate material itself or the primed substrate) can be exposed at interstitial regions between hydrogel nanoposts 46 as shown in FIG. 3.

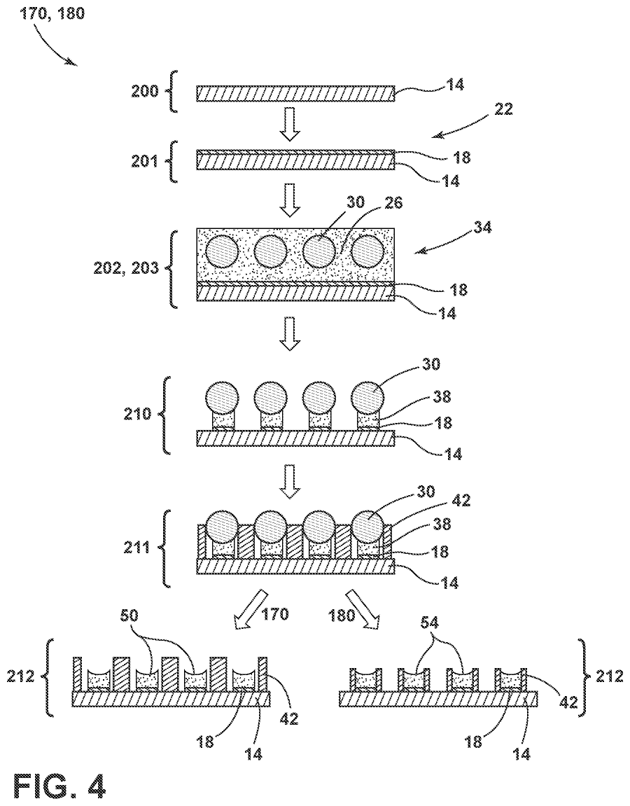

Referring to FIG. 4, a method 170 for making a periodic array of polymeric hydrogel nanoposts inside nanowells 50 is provided. The method 170 includes: providing the substrate 14 (step 200); priming the substrate 14 with the priming molecule 18 to form the primed substrate 22 (step 201); coating the primed substrate 22 with a mixture of hydrogel monomers 26 and nanoparticles 30 (e.g., depositing the mixture of hydrogel monomers and nanoparticles on the primed substrate by spin coating or another suitable deposition process) to form the coated substrate 34 (step 202); exposing the coated substrate 34 (e.g., irradiating the coated substrate with UV and/or another suitable wavelength irradiation) to form the polymeric hydrogel 38 (step 203); removing portions of the polymeric hydrogel 38 to partially expose the nanoparticles 30 and substrate 14 (210); depositing the metal layer 42, the metal oxide layer 42, or a combination thereof (e.g., depositing the metal layer, the metal oxide layer, or the combination thereof onto interstitial regions of the substrate 14 between polymeric hydrogel nanoposts and/or nanoparticles) (step 211); and etching away the nanoparticles 30 to form the periodic array of polymeric hydrogel nanoposts enclosed inside metal or metal oxide nanowells 50 (e.g., defined by the metal layer, the metal oxide layer, or the combination thereof disposed on interstitial regions of the substrate 14 between polymeric hydrogel nanoposts) (step 212). The nanoparticles can comprise inorganic nanoparticles such as, for example, silica nanoparticles. The step 211 depositing of the metal layer 42 and/or metal oxide layer 42 may be performed so the metal and/or metal oxide is deposited in the exposed space formed by the polymeric hydrogel 38 removal step 210. Each hydrogel nanopost 46 can comprise a polymeric hydrogel nanodimple as shown in FIG. 4. For example, the polymeric hydrogel nanodimple can be disposed at a top surface of the hydrogel nanopost 46.

Sill referring to FIG. 4, a method 180 for making a periodic array of polymeric hydrogel nanoposts surrounded by a metal or metal oxide ring 54 is provided. The method 180 includes: providing the substrate 14 (step 200); priming the substrate 14 with the priming molecule 18 to form the primed substrate 22 (step 201); coating the primed substrate 22 with a mixture of hydrogel monomers 26 and nanoparticles 30 (e.g., depositing the mixture of hydrogel monomers and nanoparticles on the primed substrate by spin coating or another suitable deposition process) to form the coated substrate 34 having a monolayer of non-close-packed colloidal crystals (step 202); exposing the coated substrate 34 (e.g., irradiating the coated substrate with UV and/or another suitable wavelength irradiation) to form the polymeric hydrogel 38 (step 203); removing portions of the polymeric hydrogel 38 to partially expose the nanoparticles 38 and substrate 14 (step 210); depositing the metal layer 42 and/or metal oxide layer 42 (e.g., depositing the metal layer, the metal oxide layer, or the combination thereof onto interstitial regions of the substrate 14 between polymeric hydrogel nanoposts and/or nanoparticles) (step 211); etching away a portion of the metal and/or metal oxide layer 42 (e.g., by ion beam etching or another suitable etching process) to form the metal or metal oxide ring 120; and etching away the nanoparticles 30 to form the periodic array of polymeric hydrogel nanoposts enclosed by the metal or metal oxide ring 54 (step 212). Each hydrogel nanopost 46 can comprise a polymeric hydrogel nanodimple as shown in FIG. 4. For example, the polymeric hydrogel nanodimple can be disposed at a top surface of the hydrogel nanopost 46.

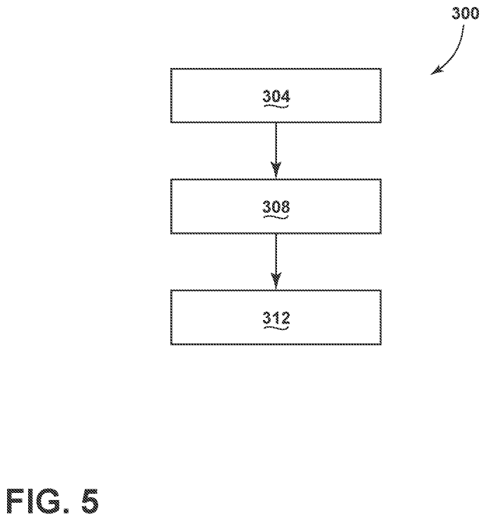

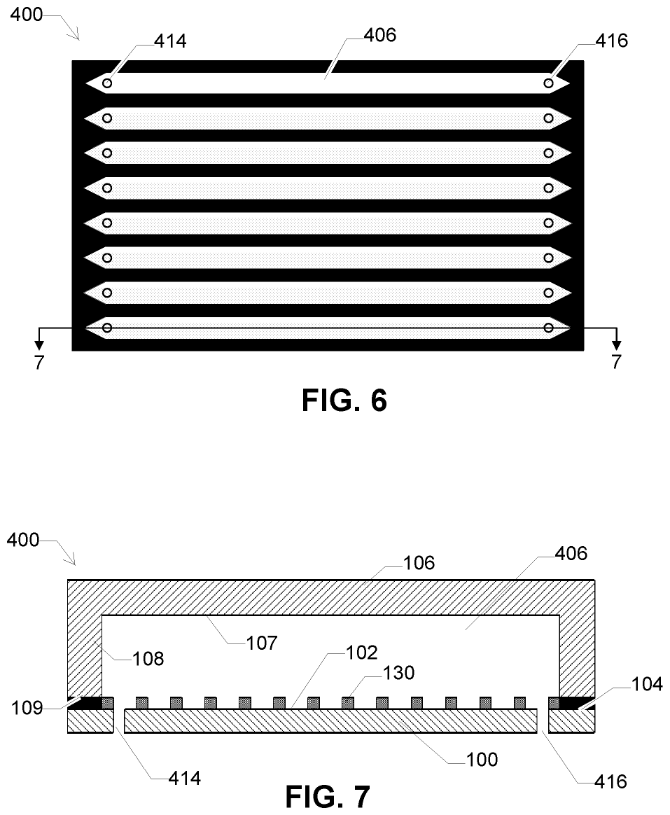

Referring to FIG. 5, a method 300 for making a microfluidic device having one or more different patterned polymeric hydrogel nanostructure 130 (see FIG. 1) is provided. FIG. 6 is a schematic top view of some embodiments of a microfluidic device 400, and FIG. 7 is a schematic cross-sectional view of the microfluidic device taken along line 7-7 of FIG. 6. The method 300 includes: providing a first substrate 100 having a first patterned array of polymeric hydrogel nanostructures 130 on a first interior surface 102 and a peripheral surface portion 104 (step 304); providing a second substrate 106 having a second interior surface 107 and a side wall 108 with an end surface 109 (step 308); and bonding the end surface 109 of the second substrate 106 to the peripheral surface portion 104 of the first substrate 100 such that the first and second interior surfaces 107 and 107 define a hermetic cavity 406 within the bonded first and second substrate (step 312). The microfluidic device 400 may include at least one fluidic channel (e.g., defined by the cavity 406). In some aspects, the microfluidic device 400 also includes at least one inlet port 414 and one outlet port 416 for each channel. Each of the channel 406, the inlet port 414, and the outlet port 416 can be formed, independently, in the pattered substrate 100 or the second substrate 106.

It is understood that the descriptions outlining and teaching the various polymeric hydrogel nanostructures 130 in FIGS. 2-4 previously discussed, which can be used in any combination, apply equally well to the method 300 of making a microfluidic device having or incorporating one or more of these different patterned polymeric hydrogel nanostructures 130.

In some aspects, the substrate 14 may be made of glass, silica, silicon, metal, ceramics, glass ceramics, or plastics. In other aspects, the solid substrate 14 may be additionally coated with a waveguide material, such as SiOₓN_{y}, Si₃N₄, Nb₂O₅, TiO₂, and Ta₂O₅. Such waveguide material coated substrates can enhance local fluorescence; in particular for total reflection fluorescence based microscopic imaging.

In some aspects, the priming molecule 18 may be a molecule that can covalently bond or attach to the substrate surface, but also react with the hydrogel monomers, in particular under UV irradiation, so that the polymeric hydrogel 38 formed can be stably attached to the substrate surface. In some aspects, the priming molecule 18 may include an acrylate silane, or a methacrylate silane, such as monoalkoxy or dialkoxy or trialkoxy acrylate or methacrylate silane. In other aspects, the priming molecule 18 may include, but is not limited to, 3-crylamidopropyltrimethoxysilane, acryloxymethyltrimethoxysilane, 3-acryloxypropyl-trichlorosilane, and/or methacryloxymethyltrimethoxysilane. In still other aspects, the priming molecule 18 may be an azide functional silane. For example, the azide functional silane may include, but is not limited to,

(azidomethyl)phenethyltrimethoxysilane, 3-azidopropyltriethoxysilane, or p-azidomethylphenyltrimethoxysilane. In still other aspects, the priming molecule 18 is an epoxy silane, such as 2-(3,4-epoxycyclohexyl)ethyltriethoxysilane, (3-glycidoxypropyl)triethoxysilane, (3-glycidoxypropyl)trimethoxysilane. In other aspects, the priming molecule 18 may include a vinyl or olefin functional silanes including, for example, 11-allyloxyundecyltrimethoxysilane, m-allylphenylpropyltriethoxysilane, allyltriethoxysilane, [(5-bicyclo[2.2.1]hept-2-enyl)ethyl]trimethoxysilane, or [2-(3-cyclohexenyl)ethyl]triethoxysilane. In again other aspects, the priming molecule 18 may include a UV active, benzophenone silane, for example, 2-hydroxy-4-(3-methyldiethoxysilylpropoxy)diphenylketone, 2-hydroxy-4-(3-triethoxysilylpropoxy)diphenylketone. In additional aspects, the priming molecule 18 may be used as an alternative UV irradiation active crosslinking agent. For example, the substrate 14 may be first coated with an amine terminated silane such as 3-aminopropyltriethoxysilane, followed by reaction with a photoactive coupling agent such as N-hydroxysulfosuccinimidyl-4-azidoenzoate (sulfo-HSAB), N-hydroxysulfosuccinimidyl-diazirine, 4-benzoylenzoic acid succinimidyl ester, and/or 4-azido-2,3,5,6-tetrafluorobenzoic acid succinimidyl ester. In some aspects, the priming molecule 18 may include an acrylate silane, an azide functional silane, a vinyl functional silane, a benzophenone silane, an amine terminated silane, or a combination thereof.

The step 201 of priming the substrate 14 with the priming molecule 18 may be performed using a solution based and/or vapor based deposition technique. For example, after cleaning the substrate 14 (e.g., glass wafers), the substrate 14 can become hydrophilic and can be primed by chemical vapor deposition of (3-acryloxypropyl)-trichlorosilane (APTCS), followed by baking at 120° C. for 30 min.

In some aspects, the mixture of hydrogel monomers 26 may include a trifunctional acrylate monomer, an acrylamide, a biomolecule binding reactive acrylamide monomer, a photoinitiator, and/or optionally an amino-acrylamide monomer. In some aspects, the trifunctional acrylate monomer may include SR 454 (ethoxylated trimethylolpropane triacrylate, ETPTA), SR351 (trimethylolpropane triacrylate), or similar reacting/functional molecules thereof. In some aspects, the biomolecule binding reactive acrylamide monomer may include N-(5-(2-bromoacetamido)pentyl)acrylamide (BRAPA) and/or N-(5-(2-azidoacetamido)pentyl)acrylamide. In some aspects, the amino-acrylamide monomer may include N-(3-aminopropyl)methacrylamide. In some aspects, the photoinitiator may include Darocur 1173 (2-hydroxy-2-methyl-1-phenyl-1-propanone) or similar reacting/functional molecules thereof. For example, in some aspects, a mixture of hydrogel monomers 26 can be made by directly dissolving acrylamide powder and BRAPA powder into ETPTA liquid. The molecular ratio among acrylamide, BRAPA, and ETPTA can be optimized based on specific requirements for biomolecular analysis or gene analysis. For example, in some aspects, the molecular ratio can be 100 ETPTA:10 acrylamide:1 BRAPA:1 N-(3-aminopropyl)methacrylamide, where the photoinitiator can be included at about 1 wt %. In some aspects, the hydrogel monomers 26 may include a trifunctional acrylate, an acrylamide, and a photoinitiator. In other aspects, the acrylamide may include a biomolecule binding reactive acrylamide monomer selected from the group consisting of N-(5-(2-bromoacetamido)pentyl)acrylamide, N-(5-(2-azidoacetamido)pentyl)acrylamide, an amino-acrylamide monomer, and a combination thereof.

In some aspects, the nanoparticles 30 can include monodispersed silica particles. In some aspects, the nanoparticles 30 may have a specific diameter from about 50 nm to about 5000 nm. In other aspects, the nanoparticles 30 may have a specific diameter from about 100 nm to about 700 nm. In still other aspects, the nanoparticles 30 may have a specific diameter from about 400 nm to about 700 nm. In other aspects, the nanoparticles 30 may have a specific diameter from about 250 nm to about 600 nm. In some aspects, the nanoparticles 30 can be fluorescent, such as by doping with a rare earth element (e.g., Eu or a similar like metal) or an organic dye (e.g., Cy3, Cy5, or the like) during the nanoparticle formation process. In some aspects, these types of fluorescent nanoparticles can be used to examine and control quality of the patterning at different steps of the manufacturing process (e.g., steps 202, 203, 204, 205, 208, 210, 211) using fluorescent microscopy, a technique which can be non-invasive.

In some aspects, the nanoparticles 30 can be dispersed into the mixture of the hydrogel monomers 26 using a variety of different mixtures. For example, commercially available monodispered silica spheres can be first purified in 200-proof ethanol using multiple centrifuge/redispersion cycles (e.g., at least 4 cycles). After complete centrifugation of the calculated amount of purified silica solution and discarding of the supernatant ethanol, the silica nanoparticles can be redispersed in a mixture of hydrogel monomers using a mixer. The final nanoparticle volume fraction may be varied to include from about 0.05 to about 0.5 or from about 0.15 to about 0.25. The particle volume fraction may be used to help determine the average distance between the particles. Due to the strong electrostatic repulsion between silica microspheres in the hydrogel monomer mixture and the refractive index matching between silica nanoparticles and the hydrogel monomers mixture (˜1.46), the silica nanoparticle/hydrogel monomer suspensions may be transparent and stable for periods of time longer than 1 month, longer than 2 months, or longer than 3 months.

In some aspects, the coating step 202 provided in FIGS. 2-4 may be used to form the coated substrate 34 having a monolayer of nonclose-packed colloidal crystals. The monolayer of nonclose-packing of colloidal crystals can be formed by spin coating the substrate 14 with a mixture of hydrogel monomers 26 and silica nanoparticles 30 where the coating process is by the electrostatic repulsion force between silica nanoparticles in combination with the sliding of nanoparticles during high-speed spinning process. For example, in some aspects, under controlled spin coating conditions, shear force-induced crystallization of silica microparticles or nanoparticles dispersed in the viscous, nonvolatile, trifunctional acrylate monomer ETPTA have been found to enable wafer-scale production of monolayer colloidal crystals having non-close-packed crystal structures (see examples discussed in Fang, Y., et al., Scalable bottom-up fabrication of colloidal photonic crystals and periodic plasmonic nanostructures. J. Materials Chem. C 2013, 1, 6031-6047). The presence of the trifunctional acrylate monomer ETPTA, after UV irradiation, can result in monolayer nonclose-packed silica colloidal crystals with controlled lattice spacing between silica particles, as well as controlled spacing between the silica nanoparticles 30 and the surface of the substrate 30. This is unlike typical nanosphere or colloidal lithography processes, where several different colloidal self-assembly techniques (e.g., spin-coating, electrical and magnetic field-induced assembly, gravity sedimentation, template-assisted assembly, and capillary force-induced self-assembly using colloidal particles in a solvent) may result in the formation of monolayer close-packed colloidal crystals, which may be further subjected, in some aspects, to a number of post-treatments such as plasma etching, pyrolysis, or electron irradiation to generate energetically unfavorable non-close-packed colloidal crystals. In some aspects, the coating obtained is a monolayer of nonclose-packed silica colloidal crystals. In some other aspects, the coating obtained is mostly a monolayer of nonclose-packed silica colloidal crystals, but also has a small portion of bilayer regions of nonclose-packed silica colloidal crystals. The presence of bilayer regions may eventually result in a relatively random feature after fully functionalized with DNA, which, in turn, can be used as a location registration, tracking or identification marker.

In some aspects, the conditions for coating (step 202) can be optimized based on the composition of the mixture of hydrogel monomers 26 and silica nanoparticle 30. For example, in some aspects, when using 330 nm silica spheres with a particle volume fraction of 0.2, the corresponding transparent colloidal suspension can be first dispensed on a primed glass wafer, where the glass wafer can then be spin-coated using a typical spin coater under a stepwise spin coating protocol, for example, at 200 rpm for 120 s, 300 rpm for 120 s, 1000 rpm for 60 s, 3000 rpm for 20 s, 6000 rpm for 20 s, and/or 8000 rpm for 360 s. The formation of wafer-sized, monolayer nonclose packed colloidal crystals on silicon or glass wafers can be indicated by the appearance of specific diffraction patterns under light illumination. For example, the diffraction pattern on silicon can be a striking six-arm Bragg diffraction star pattern. The non-close packed colloidal crystals associated with these six-arm Bragg diffraction star pattern can be embedded in the corresponding polymeric hydrogel films layered on the substrate 14. The inter-particle distance between neighboring spheres in the non-close packed colloidal crystals was found to be about 1.41× the diameter of colloids, regardless of particles sizes and spin-coating conditions when the volume fraction is kept at 0.20.

In some aspects, exposing the coated substrate 34 (e.g., with UV radiation) can result in the polymerization of the hydrogel monomers 26 into the corresponding polymeric hydrogel 38 films. For example, in some aspects, the coated substrates 34 or wafers can be exposed to a typical UV curing system such as Xenon RC-742 Pulsed UV Curing system, so that the hydrogel monomers can be photopolymerized.

In some aspects, reactive ion etching (RIE), Inductively Coupled Plasma (ICP) etching, and/or ashing can be used (see steps 204, 208, and 210) to remove the polymerized or polymeric hydrogel 38. By controlling the plasma conditions, one may control the level or amount of polymeric hydrogel removed, which, in turn, may lead to the different types of nanostructures disclosed herein. In some aspects, the polymeric hydrogel nanostructures formed can be dependent on plasma etching, metal or oxide deposition, and/or the etching process. In some aspects, a short plasma ashing using various combinations of oxygen pressure, flow rate, and power can be used to selectively remove portions of the polymeric hydrogel 38 film so the silica spheres may be partially exposed. After metal or metal oxide deposition and subsequent etching away of silica spheres (e.g., by hydrofluoric acid (HF) etching), the array of polymeric hydrogel nanodimples 10, separated by metal or oxide regions or layers in some aspects, can be formed (see FIG. 2). In other aspects, a longer plasma ashing under the same conditions can be used to completely remove the polymeric hydrogels 38 positioned between the spheres (see steps 208 and 210) but not the polymeric hydrogel 38 material positioned underneath the silica nanoparticles 30. After the silica nanoparticles are etched away using, for instance, HF, the array of polymerized hydrogel nanoposts 46 can be generated. In still other aspects, a longer plasma ashing under the same conditions can be used to completely remove the polymeric hydrogel 38 between the silica nanoparticles 30, but not the polymeric hydrogel 38 material positioned underneath the silica nanoparticles 30. After metal or metal oxide deposition in the formed open space between nanopost-silica nanoparticles and subsequent HF etching away of the silica nanoparticles 30, the array of polymerized hydrogel nanoposts enclosed inside a metal or oxide nanowell 50, can be formed. In still other aspects, a longer plasma ashing under the same condition can be used to completely remove the polymeric hydrogels 38 between the silica nanoparticles 30, but not the polymeric hydrogel 38 material positioned underneath the silica nanoparticle 30. After metal or oxide deposition, Ar ion beam etching can be used to remove the metal or oxide film, during which secondary sputtering of material creates a metal or oxide shell or ring 42 around the sides of the polymerized hydrogel materials underneath the silica nanoparticles 30. Afterwards, the silica nanoparticles 30 may be etched away using HF, so the array of polymerized hydrogel nanoposts surrounded by a metal or oxide ring 54 can be formed.

In some aspects, the metal layer 42 can include, for example, Al, Zn, Ta, Nb, Sn, Mg, Au, Ag, Ti, Ni, W, Y, Zr, Ce, Co, Cr, Fe, and In. In other aspects, the metal oxide layer 42 can include Al₂O₃, ZnO₂, Ta₂O₅, Nb₂O₅, SnO₂, MgO, indium tin oxide, CeO₂, CoO, Co₃O₄, Cr₂O₃, Fe₂O₃, Fe₃O₄, In₂O₃, Mn₂O₃, NiO, a-TiO₂ (anatase), r-TiO₂ (rutile), WO₃, Y₂O₃, ZrO₂, and/or other metal oxides. In some aspects, the metal oxide layer is transparent within a visible wavelength range (e.g., from 400 to 750 nm).

The present disclosure also includes methods to attach DNA molecules to the patterned polymeric hydrogel nanostructures on the patterned substrate 100 (see FIG. 1). In some aspects, the biomolecule binding reactive acrylamide monomer may be N-(5-(2-bromoacetamido)pentyl)acrylamide (BRAPA) so the polymeric hydrogel nanostructures obtained may include bromide functional groups. In some aspects, phosphorothioate derivatized DNA molecules can be covalently attached to the polymeric hydrogel nanostructures. For example, in some aspects, 5′-phosphorothioate oligonucleotide such as 5′-T*T*T*TTTTTTTCAAGCAGAAGACGGCATAC-3′ (*=phosphorothioate) solution in PBS buffer (pH 8.0) can be used to react with the polymeric hydrogel nanostructure patterned substrates or microfluidic devices for about 1 hr at 50° C. After the DNA molecules are blocked by 2-mercaptoethanol solution in PBS buffer (pH 8.0), the DNA molecules may covalently attach to the polymeric hydrogel nanostructures.

In some aspects, the biomolecule binding reactive acrylamide monomer can include N-(5-(2-azidoacetamido)pentyl)acrylamide, so the polymeric hydrogel nanostructures obtained can contain azido functional groups. In other aspects, alkyne modified DNA molecules can be covalently attached to the polymeric hydrogel nanostructures in the presence of N,N,N′,N′,N″-pentamethyldiethylenetriamine (PMDETA), copper sulfate, and sodium ascorbate. In still other aspects, bicyclo[6.1.0]non-4-yne terminated DNA molecules can be covalently attached to polymeric hydrogel nanostructures using UV mediated free radical reaction. In some aspects, biomolecule binding reactive acrylamide monomer may be BRAPA, but its bromide groups can be converted to azido groups using chemical reactions.

In some aspects, a method for functionalizing a microfluidic device having a patterned polymeric hydrogel nanostructure using a primer DNA is provided. The method includes providing a microfluidic device having at least one channel floor surface having a patterned polymeric hydrogel nanostructure. The method also includes incubating the microfluidic device with a primer DNA to covalently attach the primer DNA to the channel floor surface. In some aspects, the primer DNA includes a phosphorothioate derivatized primer DNA and the patterned polymeric hydrogel nanostructure includes N-(5-(2-bromoacetamido)-pentyl)acrylamide moieties. In other aspects, the incubation step includes the application of UV radiation, the primer DNA includes an alkyne modified DNA primer molecule, and the patterned polymeric hydrogel nanostructure includes N-(5-(2-azidoacetamido)pentyl)-acrylamide moieties.

It is understood that the descriptions outlining and teaching the various polymeric hydrogel nanostructures previously discussed and illustrated in FIGS. 1-4, which can be used in any combination, apply equally well to the methods of functionalizing a microfluidic device having a patterned polymeric hydrogel nanostructure using a primer DNA.

It will be understood by one having ordinary skill in the art that construction of the described device and other components may not be limited to any specific material. Other exemplary embodiments of the device disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the device as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structure without departing from the concepts of the present disclosure, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above is merely for illustrative purposes and not intended to limit the scope of the device, which is defined by the following claims as interpreted according to the principles of patent law, including the Doctrine of Equivalents.

1. A method for making a periodic array of polymeric hydrogel nanodimples, the method comprising:
priming a substrate with a priming molecule to form a primed substrate;
coating the primed substrate with a mixture of hydrogel monomers and nanoparticles to form a coated substrate;
exposing the coated substrate to form a polymeric hydrogel;
removing a portion of the polymeric hydrogel to partially expose the nanoparticles;
depositing a metal layer, a metal oxide layer, or a combination of both to a top surface of the polymeric hydrogel; and
etching away the nanoparticles to form a periodic array of polymeric hydrogel nanodimples.

2. The method of claim 1, wherein the periodic array of polymeric hydrogel nanodimples is positioned in a microfluidic device.

3. The method of claim 1, wherein the priming molecule comprises an acrylate silane, an azide functional silane, a vinyl functional silane, a benzophenone silane, an amine terminated silane, or a combination thereof.

4. The method of claim 1, wherein the hydrogel monomers comprise a trifunctional acrylate, an acrylamide, and a photoinitiator.

5. The method of claim 4, wherein the acrylamide comprises a biomolecule binding reactive acrylamide monomer selected from the group consisting of N-(5-(2-bromoacetamido)pentyl)acrylamide, N-(5-(2-azidoacetamido)pentyl)acrylamide, an amino-acrylamide monomer, and a combination thereof.

6. The method of claim 1, wherein the nanoparticles comprise silica nanoparticles with a specific diameter between about 100 nm and about 700 nm.

7. A method for making a periodic array of polymeric hydrogel nanoposts, the method comprising:
priming a substrate with a priming molecule to form a primed substrate;
coating the primed substrate with a mixture of hydrogel monomers and nanoparticles to form a coated substrate;
exposing the coated substrate to form a polymeric hydrogel;
removing portions of the polymeric hydrogel to partially expose the nanoparticles and the substrate; and
etching away the nanoparticles to form a periodic array of polymeric hydrogel nanoposts.

8. The method of claim 7, wherein the periodic array of polymeric hydrogel nanoposts is positioned in a microfluidic device.

9. The method of claim 7, wherein the priming molecule comprises an acrylate silane, an azide functional silane, a vinyl functional silane, a benzophenone silane, an amine terminated silane, or a combination thereof.

10. The method of claim 7, wherein the hydrogel monomers comprise a trifunctional acrylate, an acrylamide, and a photoinitiator.

11. The method of claim 10, wherein the acrylamide comprises a biomolecule binding reactive acrylamide monomer selected from the group consisting of N-(5-(2-bromoacetamido)pentyl)acrylamide, N-(5-(2-azidoacetamido)pentyl)acrylamide, an amino-acrylamide monomer, and a combination thereof.

12. The method of claim 7, wherein the nanoparticles comprise silica nanoparticles with a specific diameter between about 100 nm and about 700 nm.

13. A method for making a periodic array of polymeric hydrogel nanoposts enclosed inside metal or metal oxide nanowells, the method comprising:
priming a substrate with a priming molecule to form a primed substrate;
coating the primed substrate with a mixture of hydrogel monomers and nanoparticles to form a coated substrate;
exposing the coated substrate to form a polymeric hydrogel;
removing portions of the polymeric hydrogel to partially expose the nanoparticles and the substrate;
depositing a metal layer, a metal oxide layer, or a combination thereof on the exposed substrate; and
etching away the nanoparticles to form a periodic array of polymeric hydrogel nanoposts enclosed inside metal or metal oxide nanowells.

14. The method of claim 13, wherein the periodic array of polymeric hydrogel nanoposts enclosed inside the metal or metal oxide nanowells is positioned in a microfluidic device.

15. The method of claim 13, wherein the priming molecule comprises an acrylate silane, an azide functional silane, a vinyl functional silane, a benzophenone silane, an amine terminated silane, or a combination thereof.

16. The method of claim 13, wherein the hydrogel monomers comprise a trifunctional acrylate, an acrylamide, and a photoinitiator.

17. The method of claim 16, wherein the acrylamide comprises a biomolecule binding reactive acrylamide monomer selected from the group consisting of N-(5-(2-broacetamido)pentyl)acrylamide, N-(5-(2-azidoacetamido)pentyl)acrylamide, an amino-acrylamide monomer, and a combination thereof.

18. The method of claim 13, wherein the nanoparticles comprise silica nanoparticles with a specific diameter between about 100 nm and about 700 nm.

19. (canceled)

20. (canceled)

21. (canceled)

22. (canceled)

23. (canceled)

24. (canceled)

25. (canceled)

26. (canceled)

27. (canceled)

28. (canceled)
